(19) United States
(12) Patent Application Publication
(10) Pub. No.: US 2026/0047872 A1
(43) Pub. Date: Feb. 19, 2026

(54) INTRAMEDULLARY FIBULA NAIL WITH ANATOMICAL FIT, KIT, AND METHOD OF PREPARING FOR AND IMPLANTING AN INTRAMEDULLARY FIBULA NAIL

(71) Applicant: Contour MedTech, INC., Lake Zurich, IL (US)

(72) Inventors: Kyle Lappin, Lake Zurich, IL (US); Stephen McDaniel, San Rafael, CA (US); Brian Burgess, Glen Ellyn, IL (US)

(21) Appl. No.: 18/806,944

(22) Filed: Aug. 16, 2024

(51) Int. Cl. A61B 17/72; A61B 17/16

(52) U.S. Cl. CPC A61B 17/7241; A61B 17/164; A61B 17/7266; A61B 17/7283

(57) ABSTRACT

An intramedullary fibula nail kit includes an intramedullary fibula nail. The intramedullary fibula nail includes a proximal portion configured to be positioned within a medullary canal of a fibula, and a distal portion configured to be positioned within the medullary canal of the fibula. The distal portion defines a non-cylindrical cross-section.

### FIELD

The present disclosure relates generally to an intramedullary fibula nail for use in fibular fractures.

### BACKGROUND

Bone fractures are a common medical condition both in the young and old segments of the population. A wide array of implants has been developed to address the issues arising with bone fractures. Such implants include devices which provide external fixation have been developed wherein a device that protrudes out of the skin is used, and therefore carries significant risk of infection.

Other devices include bone plates which are attached directly on the bone surface. The bone plate then remains in the body long enough to allow the fractured bone to heal properly. Bone plates often require surgical exposure of a substantial length of the bone to which the plate is to be attached so as to access the bone fragments. This exposure results in a lengthy and painful healing process of the tissue around the fractured bone which is then repeated when the implantation site is again exposed to allow removal of the plate.

In some cases, an intramedullary rod or nail is used to align and stabilize the fracture. In these cases, a metal rod is placed inside a canal of a bone and fixed in place, typically at both ends. Implantation of an intramedullary nail requires incision, access to the canal, and placement of the intramedullary nail. The intramedullary nail can be subsequently removed or left in place. The outer diameter of the intramedullary nail must be selected for the minimum inside diameter of the space. Therefore, portions of the intramedullary nail may not be in contact with the bone structure surrounding the medullary canal. Lack of contact can result in micro-motion between the bone and the intramedullary nail resulting in pain or necrosis of the bone. This issue is of particular concern in devices used for fibular fractures.

There is a need for implants and related instruments that reduce at least some of the issues identified above. It would be beneficial if an intramedullary fibula nail better matched the anatomical structure of a fibula. It would be further advantageous if the intramedullary fibula nail exhibited increased resistance to rotation. Devices which simplified bone preparation for such an intramedullary nail would further be advantageous.

### SUMMARY

According to one embodiment of the present disclosure, an intramedullary fibula nail kit includes an intramedullary fibula nail with a proximal portion configured to be positioned within a medullary canal of a fibula, and a distal portion configured to be positioned within the medullary canal of the fibula. The distal portion defines a non-cylindrical geometry.

In one or more embodiments, the distal portion defines a first maximum width in a medial-lateral plane and a second maximum width in an anterior-posterior plane. The second maximum width is larger than the first maximum width and the second maximum width is larger than a maximum diameter of the proximal portion. In some of these embodiments the proximal portion is cylindrically shaped.

In one or more embodiments, the distal portion includes a neck portion which tapers to the proximal portion when the intramedullary fibula nail is implanted.

In one or more embodiments the proximal portion defines a proximal portion longitudinal axis. The distal portion defines a distal portion longitudinal axis, and the distal portion longitudinal axis is not aligned with the proximal portion longitudinal axis in the anterior-posterior plane when the intramedullary fibula nail is implanted.

In one or more embodiments an intramedullary fibula nail further includes at least one threaded anchoring structure configured to anchor the intramedullary fibula nail. The at least one threaded anchoring structure defines an activated anchoring longitudinal axis. The activated anchoring longitudinal axis is parallel to the proximal portion longitudinal axis.

In one or more embodiments the activated anchoring longitudinal axis of an anchoring structure is coaxial with the proximal portion longitudinal axis, and the proximal portion is configured to slidingly engage the distal portion.

In one or more embodiments the activated anchoring longitudinal axis of a threaded anchoring structure is not coaxial with the proximal portion longitudinal axis, and the threaded anchoring structure is configured to extend outwardly from the distal portion.

In one or more embodiments the intramedullary fibula nail further includes at least one serrated anchoring structure configured to anchor the intramedullary fibula nail. The proximal portion defines a proximal portion longitudinal axis. The at least one serrated anchoring structure defines at least one activated anchoring longitudinal axis, and the at least one activated anchoring longitudinal axis is parallel to the proximal portion longitudinal axis.

In one or more embodiments the intramedullary fibula nail includes at least one rod configured to extend outwardly from the distal portion and to support the at least one serrated anchoring structure.

In one or more embodiments the intramedullary fibula nail includes at least one suture activated anchoring structure, and an activation suture operably connected to the at least one suture activated anchoring structure.

In one or more embodiments the distal portion defines a suture cross-over region. The intramedullary fibula nail kit includes a clamping screw, and the clamping screw is configured to clamp the activation suture within the suture cross-over region.

In one or more embodiments the proximal portion defines a proximal portion longitudinal axis, and the activation suture is configured to bias at least a portion of the at least one suture activated anchoring structure in a direction away from the proximal portion longitudinal axis when the activation suture is activated.

In one or more embodiments the activation suture is operably connected to the at least one suture activated anchoring structure through a wedge component.

In one or more embodiments the at least one suture activated anchoring structure is pivotably connected to the proximal portion through at least one pivot pin.

In one or more embodiments the at least one suture activated anchoring structure comprises two suture activated anchoring structures, each of the two suture activated anchoring structures pivotably connected to the proximal portion through a respective one of the at least one pivot pin.

In one or more embodiments a retraction suture is operably connected to the at least one suture activated anchoring structure and configured to bias at least a portion of the at least one suture activated anchoring structure in a direction toward the proximal portion longitudinal axis when the retraction suture is activated.

In one or more embodiments the kit further includes a guide reamer including a sleeve coupling portion and at least one side guide channel, and a guide sleeve including a central coupling portion configured to non-rotatably couple with the sleeve coupling portion of the guide reamer, the guide sleeve including at least one offset guide channel. The kit further includes at least one offset reamer. The at least one offset guide channel and the at least one side guide channel are configured to guide the at least one offset reamer within the guide sleeve and the at least one side guide channel is configured to guide the at least one offset reamer outside of the guide sleeve.

In one or more embodiments the kit further includes a plurality of screws, and the distal portion includes a plurality of elongated screw holes. The plurality of screws and the plurality of elongated screw holes are configured to provide a syndesmosis fixation angle of between 10 and 50 degrees. More preferably, the plurality of screws and the plurality of elongated screw holes are configured to provide a syndesmosis fixation angle of between 20 and 45 degrees. In one embodiment the plurality of screws and the plurality of elongated screw holes are configured to provide a syndesmosis fixation angle of about 40 degrees.

In accordance with one embodiment of the disclosure a method of preparing a fibula for an intramedullary fibula nail includes accessing a distal portion of the fibula. The method includes reaming the fibula through the accessed distal portion with a guide reamer including a sleeve coupling portion and a side guide channel, and non-rotatably coupling a central coupling portion of a guide sleeve to the sleeve coupling portion of the guide reamer. A side reamer is then inserted into an offset guide channel of the non-rotatably coupled guide sleeve and guided with the side guide channel and the offset guide channel within the non-rotatably coupled guide sleeve. The method includes reaming the accessed distal portion with the inserted side reamer while guiding the inserted side reamer outside of the guide sleeve with the side guide channel.

In accordance with one embodiment of the disclosure a method of implanting an intramedullary fibula nail includes preparing a fibular medullary canal for implantation. The method continues by selecting an intramedullary fibula nail with a proximal portion, and a distal portion that exhibits a non-cylindrical geometry, and implanting the distal portion within the fibular medullary canal.

In one or more embodiments of the method of implanting an intramedullary fibula nail the selected intramedullary fibula nail includes a distal portion which defines a first maximum width in a medial-lateral plane and a second maximum width in an anterior-posterior plane. The second maximum width is larger than the first maximum width, and the method further comprises activating an anchoring structure after implanting the distal portion.

### BRIEF DESCRIPTION OF THE DRAWINGS

The above-described features and advantages, as well as others, will become more readily apparent to those of ordinary skill in the art by reference to the following detailed description and accompanying drawings.

FIG. 1 depicts perceptive or plan views of components of an intramedullary fibula nail kit including intramedullary fibula nails, reamers, and guides;

FIG. 2 depicts a distal perspective view of one of the intramedullary fibula nails of FIG. 1 along with various screws included in the intramedullary fibula nail kit;

FIG. 3 depicts a plan view of the distal portion of the intramedullary fibula nail of FIG. 2;

FIG. 4 depicts a cross-sectional view of the intramedullary fibula nail of FIG. 2 showing a clamping screw which is included in the kit of FIG. 1;

FIG. 5 depicts a cross-sectional view of the suture activated anchoring structure of the intramedullary fibula nail of FIG. 2 with the suture activated anchoring structure in a stowed configuration;

FIG. 6 depicts a cross-sectional view of the suture activated anchoring structure of the intramedullary fibula nail of FIG. 2 with the suture activated anchoring structure in an activated configuration;

FIG. 7 depicts a side perspective view of one of the intramedullary fibula nails of FIG. 1;

FIG. 8 depicts a cross-sectional view of the intramedullary fibula nail of FIG. 7;

FIG. 9 depicts a cross-sectional view of the suture activated anchoring structure of the intramedullary fibula nail of FIG. 7 with the suture activated anchoring structure in a stowed configuration;

FIG. 10 depicts a cross-sectional view of the suture activated anchoring structure of the intramedullary fibula nail of FIG. 7 with the suture activated anchoring structure in an activated configuration;

FIG. 11 depicts a side perspective view of one of the intramedullary fibula nails of FIG. 1;

FIG. 12 depicts a side plan view of the intramedullary fibula nail of FIG. 11;

FIG. 13A depicts a side plan view of an alternative embodiment of a suture activated anchoring structure of one of the intramedullary fibula nails of FIG. 1 with the suture activated anchoring structure in a stowed configuration;

FIG. 13B depicts a side plan view of an alternative embodiment of a suture activated anchoring structure of one of the intramedullary fibula nails of FIG. 1 with the suture activated anchoring structure in an activated configuration;

FIG. 14 depicts a side plan view of an alternative embodiment of a suture activated anchoring structure of one of the intramedullary fibula nails of FIG. 1 with the suture activated anchoring structure in a stowed configuration;

FIG. 15 depicts a perspective view of an alternative embodiment of a suture activated anchoring structure of one of the intramedullary fibula nails of FIG. 1 with the suture activated anchoring structure in a stowed configuration;

FIG. 16 depicts a perspective view of the suture activated anchoring structure of FIG. 15 with the suture activated anchoring structure in an activated configuration;

FIG. 17 depicts a perspective view of an alternative embodiment of a suture activated anchoring structure of one of the intramedullary fibula nails of FIG. 1 with the suture activated anchoring structure in a stowed configuration;

FIG. 18 depicts a side plan view of the alternative embodiment of a suture activated anchoring structure of FIG. 17 with the suture activated anchoring structure in a stowed configuration;

FIG. 19 depicts a side plan view of an alternative embodiment of a suture activated anchoring structure of one of the intramedullary fibula nails of FIG. 1 with the suture activated anchoring structure in an activated configuration;

FIG. 20 depicts a cross-sectional view of an alternative embodiment of a suture activated anchoring structure of one of the intramedullary fibula nails of FIG. 1 with the suture activated anchoring structure in an activated configuration;

FIG. 21 depicts a side perspective view of one of the intramedullary fibula nails of FIG. 1;

FIG. 22 depicts a cross-sectional view of the distal portion of the intramedullary fibula nail of FIG. 21;

FIG. 23 depicts a side perspective view of one of the intramedullary fibula nails of FIG. 1;

FIG. 24 depicts a perspective view of the proximal portion of the intramedullary fibula nail of FIG. 23;

FIG. 25 depicts a cross-sectional view of the anchoring structure of the intramedullary fibula nail of FIG. 23 with the anchoring structure in a stowed configuration;

FIG. 26 depicts a cross-sectional view of the anchoring structure of the intramedullary fibula nail of FIG. 23 with the anchoring structure in an activated configuration;

FIG. 27 depicts a perspective view of one of the intramedullary fibula nails of FIG. 1;

FIG. 28 depicts a perspective view of the intramedullary fibula nail of FIG. 27;

FIG. 29 depicts a cross-sectional view of the anchoring structure of the intramedullary fibula nail of FIG. 27;

FIG. 30 depicts a side plan view of the distal portion of an intramedullary fibula nail of FIG. 1;

FIG. 31 depicts a bottom plan view of the distal portion of the intramedullary fibula nail of FIG. 30;

FIG. 32A depicts a cross-sectional view of a fibula and a tibia;

FIGS. 32B and 32C depict views of the fibula and tibia of FIG. 32A along with or overlaid with screws, intramedullary fibula nails, and or bone plates provided in various embodiments of the kit of FIG. 1 showing a range of syndesmosis fixation angles available with the wider anterior-posterior distal structure provided by the intramedullary fibula nails of FIG. 1;

FIG. 33 depicts a schematic bottom plan view of the distal portion of the intramedullary fibula nail of FIG. 30 within a medullary canal of a fibula;

FIG. 34 depicts a schematic plan view in a medial-lateral plane of the intramedullary fibula nail of FIG. 30 within a medullary canal of a fibula;

FIG. 35 depicts a schematic plan view in an anterior-posterior plane of the intramedullary fibula nail of FIG. 30 within a medullary canal of a fibula;

FIG. 36 depicts schematic bottom plan views of non-cylindrical distal portion cross-sectional shapes for the intramedullary fibula nails of FIG. 1, along with schematics of reamer diameters used to provide cavities in a medullary canal of a fibula to receive the non-cylindrical distal portion cross-sectional shapes;

FIG. 37 depicts a side perspective view of the guide reamer of the kit of FIG. 1;

FIG. 38 depicts a rear plan view of the guide reamer of FIG. 37;

FIG. 39 depicts a side perspective view of the guide sleeve of the kit of FIG. 1;

FIG. 40 depicts a rear plan view of the guide sleeve of FIG. 39 coupled with the guide reamer of FIG. 37;

FIG. 41 depicts a perspective view of side reamers provided with the kit of FIG. 1;

FIG. 42 depicts a front perspective view of the guide sleeve of FIG. 39 coupled with the guide reamer of FIG. 37 with the side reamers of FIG .41 guided by the guide sleeve of FIG. 39 and the guide reamer of FIG. 37;

FIG. 43 depicts a side plan view of the guide sleeve of FIG. 39 coupled with the guide reamer of FIG. 37 with the side reamers of FIG. 41 guided by the guide sleeve of FIG. 39 and the guide reamer of FIG. 37;

FIG. 44 depicts a process for preparing a medullary canal of a fibula using components in the kit of FIG. 1;

FIG. 45 depicts a process of implanting an intramedullary fibula nail from the kit of FIG. 1 in a prepared medullary canal of a fibula;

FIGS. 46-47 depict perspective views of a combined guide reamer and sleeve which in some embodiments I included in the kit of FIG. 1; and

FIGS. 48-49 depict cross-sectional views of an intramedullary fibula nail incorporating a nitinol tensioner used in one or more of the intramedullary fibula nails in the kit of FIG. 1 which is configured to maintain tension on a wedge structure of a suture activated anchoring structure after implantation of the intramedullary fibula nail.

### DETAILED DESCRIPTION

For the purpose of promoting an understanding of the principles of the disclosure, reference will now be made to the embodiments illustrated in the drawings and described in the following written description. It is to be understood that no limitation to the scope of the disclosure is thereby intended. It is further to be understood that the present disclosure includes any alterations and modifications to the illustrated embodiments and includes further applications of the principles of the disclosure as would normally occur to one skilled in the art to which this disclosure pertains.

The present disclosure uses as a frame of reference planes which are based upon anatomical planes. Accordingly, the median plane is referred to herein as the “anterior-posterior plane”, and the coronal plane is referred to herein as the “medial-lateral plane”. The terms “distal” and “proximal” as used herein refer to location with respect to the transverse plane.

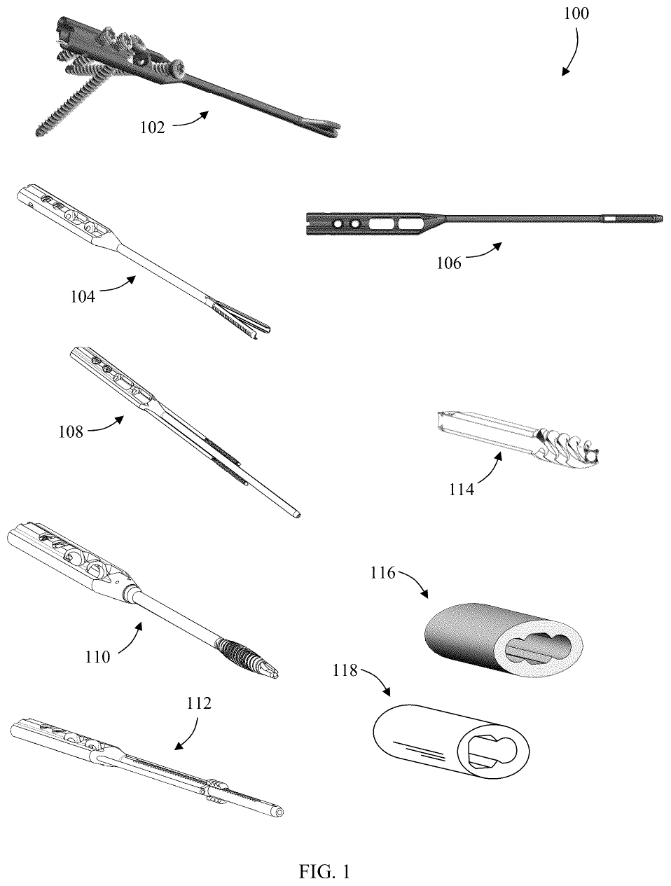

FIG. 1 depicts an intramedullary fibula nail kit 100 including intramedullary fibula nails 102, 104, 106, 108, 110, and 112. While only one size of each of the nails is shown, the kit 100 in some embodiments includes a variety of differently sized intramedullary fibula nails of some or all of the intramedullary fibula nails 102, 104, 106, 108, 110, and 112. Additionally, in some embodiments the kit 100 includes only one or a subset of the intramedullary fibula nails 102, 104, 106, 108, 110, and 112. The kit 100 further includes a guide reamer 114 and guide sleeves 116 and 118. In some embodiments the kit 100 includes differently sized guide reamers and guide sleeves which are associated with differently sized intramedullary fibula nails.

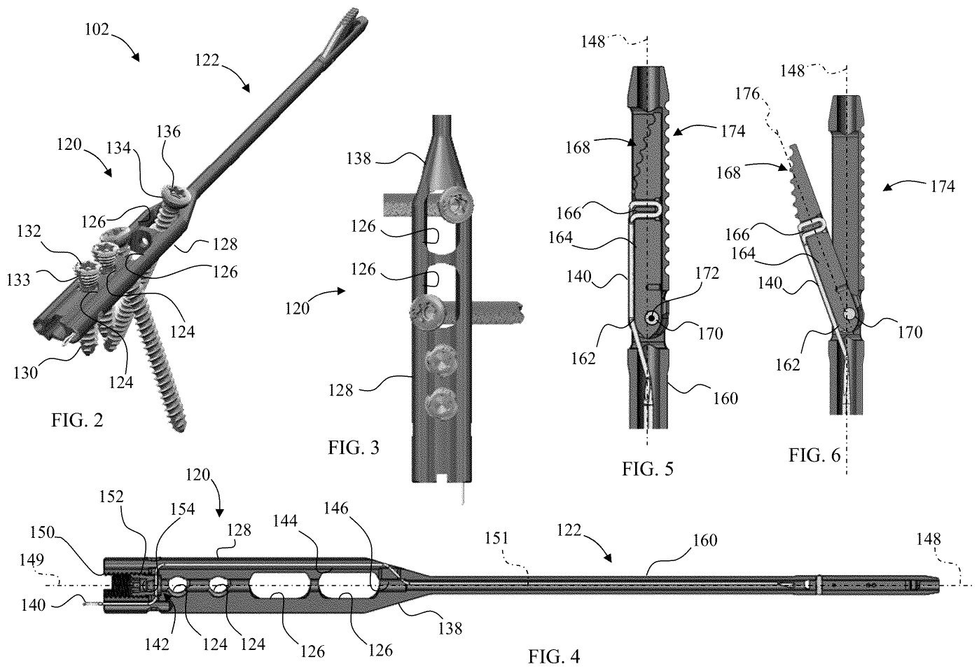

The intramedullary fibula nail 102, shown in more detail in FIGS. 2-6, includes a distal portion 120 and a proximal portion 122. The distal portion 120 is configured to be positioned within a medullary canal of a fibula and includes threaded screw holes 124 and elongated screw holes 126 within a body portion 128. The threading in the threaded screw holes 122 is configured to receive threaded shanks 130 of screws 132 so as to fix the screws 132 in a predetermined orientation with respect to the distal portion 120. The screws 132 further include head threads 133 which allow the screw to threadedly engage the lateral cortex so that the screws are sub-flush with the bone and have increased boney engagement compared to countersinking heads. The elongated screw holes 126 are configured to receive spherical heads 134 of screws 136 at a variety of relative orientations to provide flexibility in anchoring the distal portion 120 to a fibula. The screws 132 and the screws 136 are included in the kit 100. The distal portion includes a neck portion 138 which tapers from the body portion 128 to the proximal portion 122.

As shown in FIG. 4, an activation suture 140 extends distally from the distal portion 120. The activation suture 140 extends through a suture cross-over region 142 within the body portion 128 and along a suture passage 144 which leads to a guide bore 146 which extends along a longitudinal axis 148 of the intramedullary fibula nail 102. The longitudinal axis 149 of the distal portion 120 and the longitudinal axis 151 of the proximal portion 122 in this embodiment are coaxial with the longitudinal axis 148.

The body portion 128 further includes a threaded bore 150 (see FIG. 4) which opens to the suture cross-over region 142. A clamping screw 152, provided with the kit 100, is positionable within the threaded bore 150 so as to selectively clamp the suture 140 within the suture cross-over region 142. The clamping screw 152 includes an internal through-bore 154 which is aligned with the longitudinal axis 148, and hence the guide bore 146, when the clamping screw 152 is threadedly engaged with the threaded bore 150.

The suture 140 passes through the tapered neck portion 138 and through a body portion 160 of the proximal portion 122. The proximal portion 122 is configured to be implanted in a medullary canal of a fibula. As shown in FIG. 5, the suture passes over a shoulder 162 of a suture activated anchoring structure 164 and is fixedly connected to a coupling structure 166 of the suture activated anchoring structure 164.

The suture activated anchoring structure 164 includes a serrated portion 168 located proximally of the coupling structure 166. The suture activated anchoring structure 164 is pivotably connected to the body portion 160 by a pivot pin 170. The pivot pin 170 defines a pivot axis 172 which is spaced apart from the longitudinal axis 148 on a side of the longitudinal axis opposite the shoulder 162. A serrated portion 174 of the body portion 160 extends along the axis 148 generally opposite the serrated portion 168.

The suture activated anchoring structure 164 is configured such that when suture 140 is activated, the suture 140 moves distally with the shoulder 162 acting as a fulcrum such that the suture activated anchoring structure 164 pivots about the pivot axis 172 such that the serrated portion 168 of the suture activated anchoring structure 164 moves outwardly away from the longitudinal axis 148 from the stowed position of FIG. 5 to the activated position of FIG. 6. While the axis of the suture activated anchoring structure 164 is generally aligned with the longitudinal axis 148 in the stowed position of FIG. 5, as shown in FIG. 6, the suture activated anchoring structure 164 defines an activated anchoring longitudinal axis 176 which is not parallel to the longitudinal axis 148 of the intramedullary fibula nail 102 when activated.

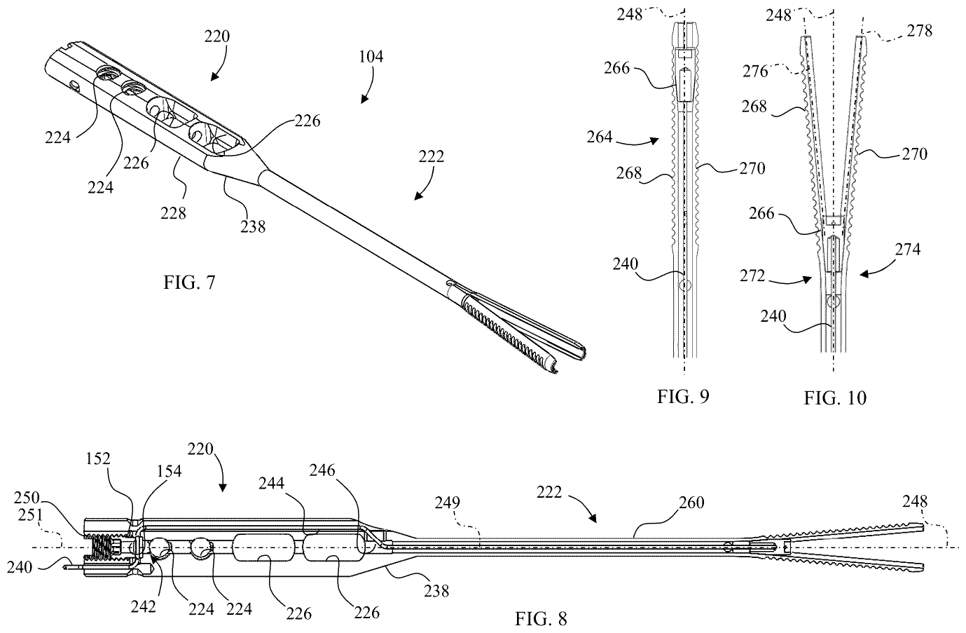

The intramedullary fibula nail 104, shown in more detail in FIGS. 7-10, includes a distal portion 220 and a proximal portion 222. The distal portion 220 is configured to be positioned within a medullary canal of a fibula and includes threaded screw holes 224 and elongated screw holes 226 within a body portion 228. The threading in the threaded screw holes 222 is configured to receive the threaded shank 130 of screws 132 shown in FIG. 2 so as to fix the screws 132 in a predetermined orientation with respect to the distal portion 220. The elongated screw holes 226 are configured to receive spherical heads 134 of screws 136 shown in FIG. 2 at a variety of relative orientations to provide flexibility in anchoring the distal portion 220 to a fibula. The distal portion includes a neck portion 238 which tapers from the body portion 228 to the proximal portion 222.

As shown in FIG. 8, an activation suture 240 extends distally from the distal portion 220. The activation suture 240 extends through a suture cross-over region 242 within the body portion 228 and along a suture passage 244 which leads to a guide bore 246 which extends along a longitudinal axis 248 of the intramedullary fibula nail 104. The longitudinal axis 249 of the proximal portion 222 and the longitudinal axis 251 of the distal portion 220 in this embodiment are coaxial with the longitudinal axis 248.

The body portion 228 further includes a threaded bore 250 which opens to the suture cross-over region 242. The clamping screw 152, provided with the kit 100, is positionable within the threaded bore 250 so as to selectively clamp the suture 240 within the suture cross-over region 242. The internal through-bore 154 of the clamping screw 152 is aligned with the longitudinal axis 248, and hence the guide bore 246, when the clamping screw 152 is threadedly engaged with the threaded bore 250. In some embodiments a different structure is incorporated to provide locking of the suture. By way of example, in one embodiment a clamping component clamps the suture and is wedged against the distal portion. In other embodiments, the suture is tied to a locking structure.

The suture 240 passes through the tapered neck portion 238 and through a body portion 260 of the proximal portion 122. The proximal portion 122 is configured to be implanted in a medullary canal of a fibula. As shown in FIG. 9, the suture is fixedly connected to a wedge structure 266 of a suture activated anchoring structure 264.

The suture activated anchoring structure 264 includes a serrated arm portion 268 and a serrated arm portion 270. The suture activated anchoring structure 264 is configured such that when suture 240 is activated, the suture 240 moves distally forcing the wedge structure 266 against the serrated arm portions 268/270. Pivot regions 272/274, which are provided by the flexibility of the material used in forming the serrated arm portions 268/270 (see FIG. 10), allow the proximal ends of the serrated arm portions 268/270 to move outwardly away from the longitudinal axis 248 from the stowed position of FIG. 9 to the activated position of FIG. 10. While the axes of the suture activated anchoring structure 264 defined by the serrated arm portions 268/270 are parallel to the longitudinal axis 248 in the stowed position of FIG. 9, as shown in FIG. 10, the suture activated anchoring structure 264 defines two activated anchoring longitudinal axes 276/278 which are not parallel to the longitudinal axis 248 of the intramedullary fibula nail 104 when activated.

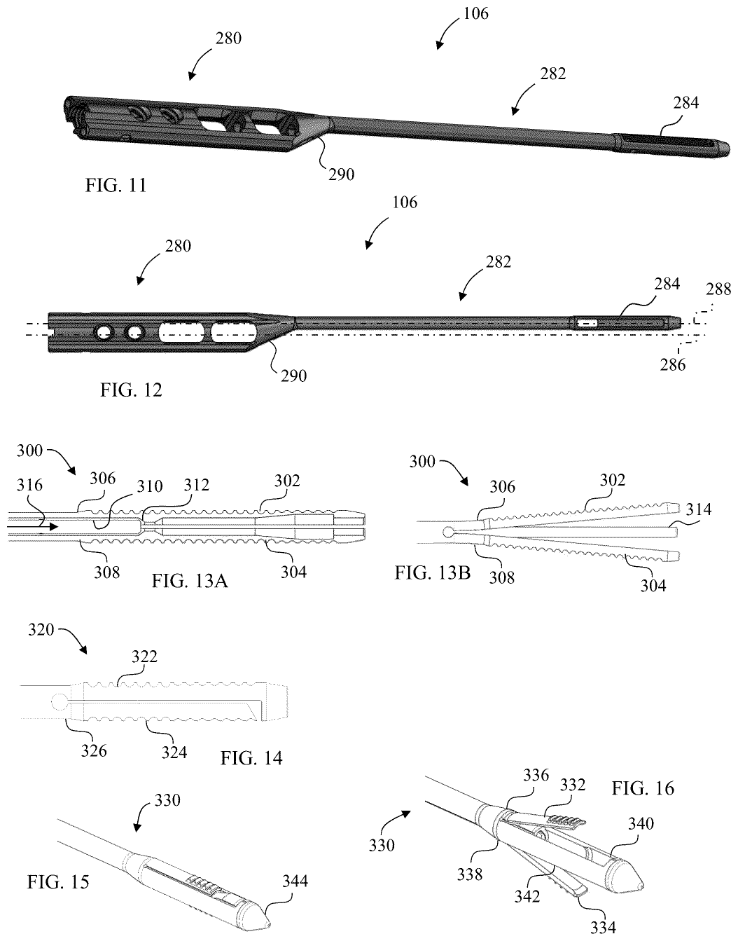

The intramedullary fibula nail 106, also shown in FIGS. 11 and 12, is configured like the intramedullary fibula nail 102 including a distal portion 280 and a proximal portion 282 with a suture activated anchoring structure 284. The difference is that a longitudinal axis 286 of the distal portion 280 is offset from a longitudinal axis 288 of the proximal portion 282 in the anterior-posterior plane as shown in FIG. 12.

Accordingly, the taper of the neck portion 290 is configured differently as shown in FIGS. 11 and 12. Due to the offset in the longitudinal axes 286/288, the configuration of the intramedullary fibula nail 106 is useful in reducing fractures by leveraging the bone fragments into alignment with the intramedullary fibula nail 106.

By way of example, if one of the other intramedullary fibula nails in the kit 100 is initially manipulated which leads to undesired movement of a bone segment anteriorly or posteriorly, then the intramedullary fibula nail 106 is substituted to reduce the fracture with the geometry of the intramedullary fibula nail 106. Accordingly, the kit 100 in some embodiments includes the intramedullary fibula nail 106 and another the intramedullary fibula nail with the distal portion 280 offset from the longitudinal axis 288 in an opposite direction to the intramedullary fibula nail 106.

While various suture activated anchoring structures are shown with the intramedullary fibula nails 102, 104, and 106, in other embodiments one or more of the intramedullary fibula nails 102, 104, and 106 are provided with suture activated anchoring structures shown in FIGS. 13-20. In FIG. 13A, the proximal portion 300 includes an anchoring structure including serrated arm portions 302/304 and associated pivot regions 306/308 like those shown in FIGS. 9 and 10. The proximal portion 300 further includes, however, an activation channel 310 which terminates in an activation neck 312. To activate the anchoring structure of FIG. 13A, a rod 314 (see FIG. 13B) is inserted in the activation channel 310 in the direction of the arrow 316. When the rod 314 contacts the activation neck 312, the serrated arm portions 302/304 are forced to pivot outwardly to the activated position shown in FIG. 13B.

The proximal portion 320 in FIG. 14 includes fixed anchoring structure 322 and a suture activated anchoring structure 324 with an associated pivot region 326. The suture activated anchoring structure 324 is wedge activated like the serrated arm portion 304 thus providing a single activatable structure.

The proximal portion 330 shown in FIGS. 15-16 includes serrated arm portions 332/334 and associated pivot regions 336/338 like those shown in FIGS. 9 and 10. The proximal portion 330 further includes, however, two sidewalls 340/342 which extend along the serrated arm portions 332/334 and support a tip portion 344. The tip portion 344 is shown without a guide bore, but in some embodiments a guide bore is provided.

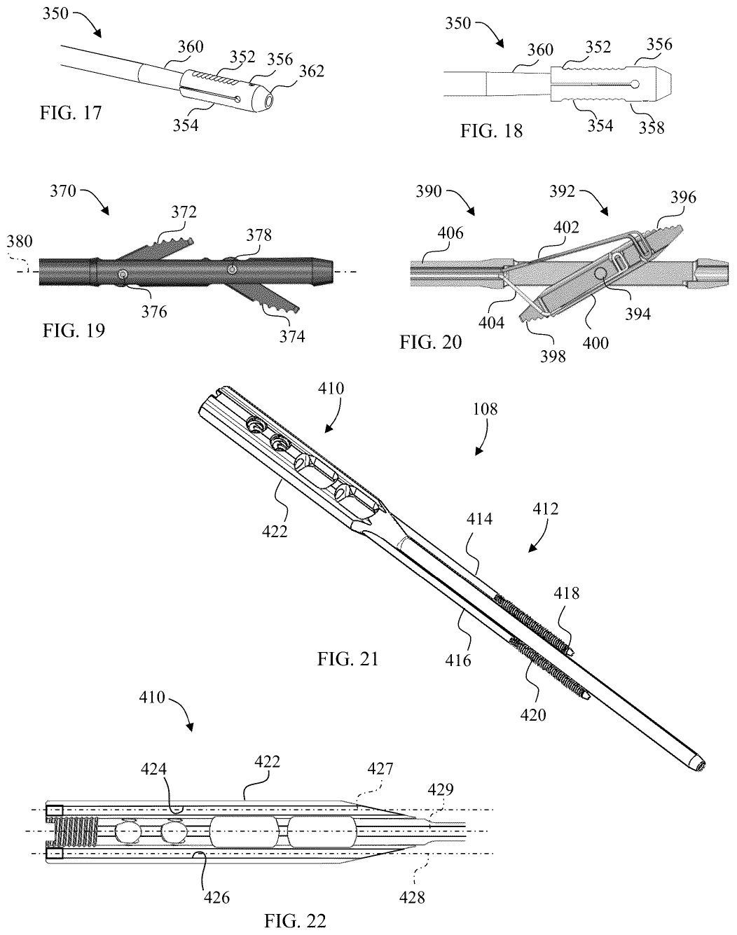

The proximal portion 350 shown in FIGS. 17-18 includes serrated arm portions 352/354 and associated pivot regions 356/358 similar those shown in FIGS. 9 and 10. The proximal portion 350 further includes, however, a stationary wedge structure 360 which supports the serrated arm portions 352/354 which are joined by a tip portion 362. The suture (not shown) is attached to the tip portion 362 such that activation of the suture pulls the tip portion 362 distally such that the wedge structure 360 forces the serrated arm portions 352/354 to pivot outwardly.

The proximal portion 370 shown in FIG. 19 includes serrated arm portions 372/374 and associated pivots 376/378, each of which is similar the one shown in FIGS. 5 and 6. Unlike the configuration of FIGS. 5 and 6, however, the proximal portion 370 provides suture activated anchoring structures which are spaced along the longitudinal axis 380 of the proximal portion 370. The serrated arm portions 372/374 in one embodiment are activated by a single suture operably connected to each of the serrated arm portions 372/374. In another embodiment, only the serrated arm portion 372 is directly activated by the suture and the serrated arm portion 374 is activated by the activation of the serrated arm portion 372.

The proximal portion 390 shown in FIG. 20 includes a suture activated anchoring structure 392 and associated pivot 394 which is similar the one shown in FIGS. 5 and 6. Unlike the configuration of FIGS. 5 and 6, however, suture activated anchoring structure 392 includes two serrated arm portions 396/398 located at opposite ends of an anchoring structure arm 400. An activation suture 402 activates the suture activated anchoring structure 392 like the activation suture 140. Because the serrated arm portions 396/398 are located at opposite ends of an anchoring structure arm 400 and on opposite sides of the pivot 304, however, the serrated arm portions 396/398 cannot be released simply by unclamping the activation suture 402 and moving the proximal portion 390 distally as is the case with the configuration of FIGS. 5 and 6. Consequently, a retraction suture 404 is provided which moves the anchoring structure arm 400 from the activated position shown in FIG. 20 to a stowed position (not shown) with the anchoring structure arm 400 aligned with the body 406 of the proximal portion 390.

The intramedullary fibula nail 108 which is shown in FIG. 21 includes a distal portion 410 and a proximal portion 412. Intramedullary fibula nail 108 is similar to the intramedullary fibula nails 102, 104, and 106 but does not include a suture or a suture activated anchoring structure. Proximal anchoring of the intramedullary fibula nail 108 is provided by two locking screws 414/416. The locking screws 414/416 include bone thread portions 418/420 which are configured to be threaded into bone of the fibula to anchor the intramedullary fibula nail 108. To accommodate the threaded heads of the locking screws 414/416 the body portion 422 includes two threaded through-bores 424/426 shown in FIG. 22. The activated anchoring longitudinal axes 427/428 respectively defined by the locking screws 414/416 and the respective bone thread portions 418/420 are parallel to, but offset from, the longitudinal axis 429 of the intramedullary fibula nail 108 when the bone thread portions 418/420 are activated by the rotation of the locking screws 414/416. The stowed anchoring longitudinal axes are coaxial with the activated anchoring longitudinal axes 427/428.

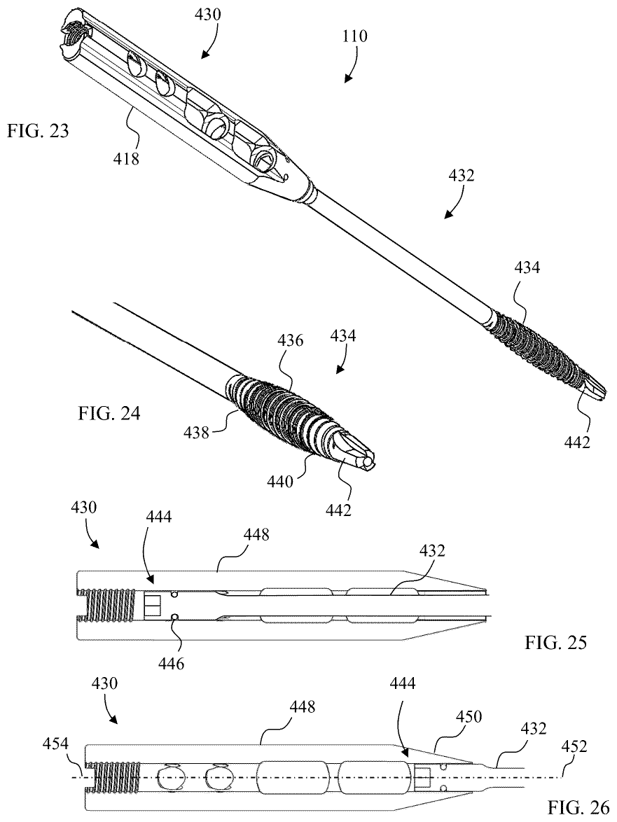

The intramedullary fibula nail 110 which is shown in FIG. 23 includes a distal portion 430 and a proximal portion 432. Like the intramedullary fibula nail 108, the intramedullary fibula nail 110 uses a threaded anchor structure to anchor the intramedullary fibula nail 110. To this end, the proximal portion 432 includes a bone thread portion 434 shown in FIG. 24. The bone thread portion 434 includes an expanded central region 436 and tapered portions 438/440. A reamer portion 442 is provided proximally to the bone thread portion 434.

In this embodiment the distal portion 430 and the proximal portion 432 are separately formed. As shown in FIG. 25, the proximal portion 432 includes a coupling portion 444 and a sealing gasket 446. The coupling portion 444 is configured to couple with a manual or motorized rotation device (not shown). Consequently, the intramedullary fibula nail 110 can be positioned in a stowed configuration as shown in FIG. 25.

In FIG. 25, the coupling portion 444 is positioned within the body 448 of the distal portion 430. Once the distal portion 430 with the stowed proximal portion 432 is implanted in a fibula, the proximal portion 432 is rotated using the coupling portion 444. This causes the reamer portion 442 to ream the medullary canal of a fibula while the bone thread portion 434 engages the fibula. The shape of the bone thread portion 434 allows the bone thread portion 434 to burrow into the medullary canal pulling the proximal portion 432 out of the body portion 448 until the coupling portion 444 is located within the tapered neck portion 450. As shown in FIG. 26, the anchoring longitudinal axis 452 defined by the proximal portion 432 and the bone thread portion 434 is parallel to, and coaxial with, the longitudinal axis 454 of the distal portion 430. The stowed anchoring longitudinal axis is coaxial with the activated anchoring longitudinal axis.

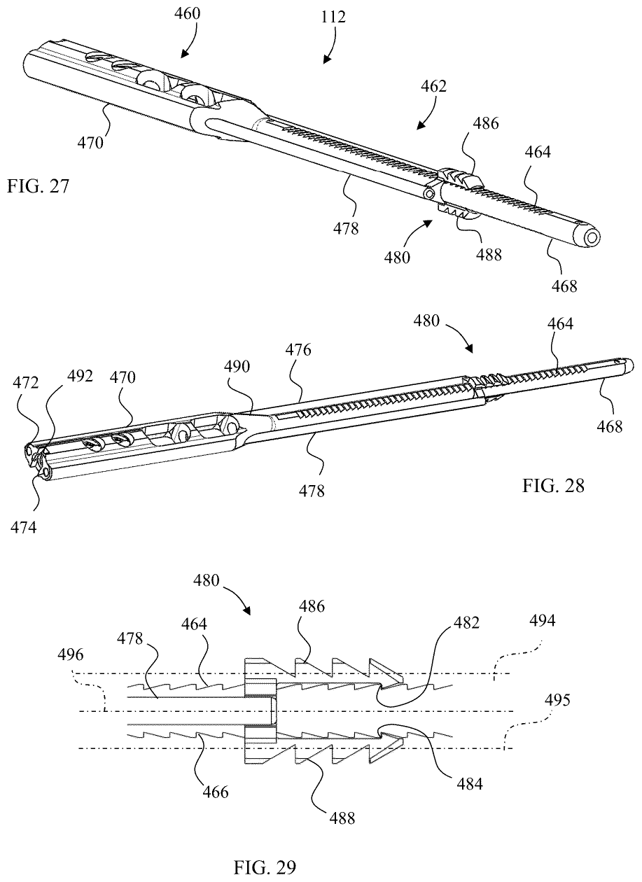

The intramedullary fibula nail 112 is shown in FIGS. 27 and 28. The intramedullary fibula nail 112 includes a distal portion 460 and a proximal portion 462. The proximal portion 462 includes a pair of toothed racks 464/466 positioned on opposite sides of the body 468 as shown in FIG. 29. The body 470 of the distal portion 460 includes two through-bores 472/474 which slidingly support rods 476/478. The rods 476/478 support a pawl block 480 which includes pawl teeth 482/484 at the inner end portions of serrated arm portions 486/488, respectively. The anchoring longitudinal axes 494/495 respectively defined by the serrated arm portions 486/488 are parallel to, and offset from, the longitudinal axis 496 of the intramedullary fibula nail 112. The stowed anchoring longitudinal axes are coaxial with the activated anchoring longitudinal axes.

During implantation, the pawl block 480 is initially positioned close to the neck 490 in a stowed position. Once the distal and proximal portions 460/462 are positioned within a medullary canal of a fibula, the rods 476/478 are used to force the pawl block 480 proximally. The serrated arm portions 486/488 flex to allow the pawl teeth 482/484 to slide over the teeth of the toothed racks 464/466 as the rods 476/478 and pawl block 480 are moved proximally.

In some embodiments, the through-bores 472/474 are threaded and the rods 476/478 include threaded heads which engage the through-bores 472/474 to lock the rods 476/478 to the body 470. In other embodiments the clamping screw 152 is inserted into the threaded bore 492 (see FIG. 28) to activate a clamping mechanism (not shown) to lock the rods 476/478 to the body 470.

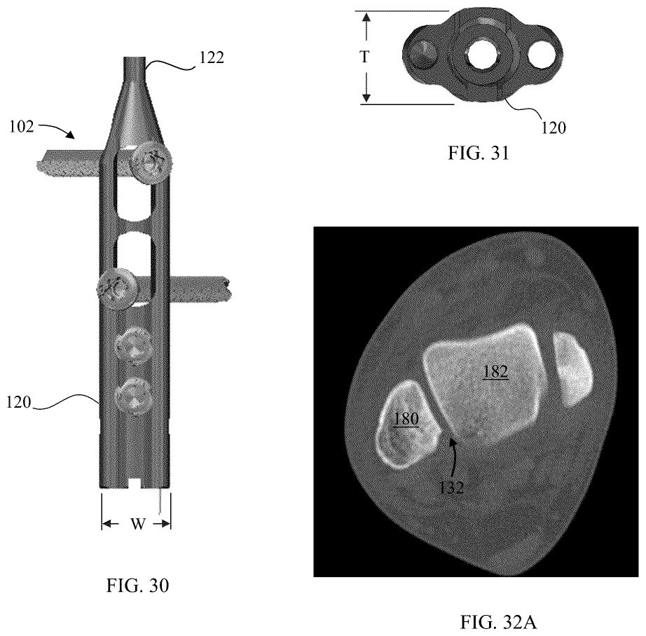

In each of the intramedullary fibula nails of FIG. 1 the basic construct of the proximal and distal portions are similar as discussed with respect to the intramedullary fibula nail 102 shown in FIGS. 30 and 31. While the proximal portion 122 is generally cylindrical, the distal portion 120 is non-cylindrical. In some embodiments the distal portion 120 has a width (W) in the anterior-posterior plane which is greater than the thickness (T) in the medial-lateral plane. This provides rotational control of the distal fragment without screws or pins, and allows for variable angle syndesmotic fixation using the elongated screw holes.

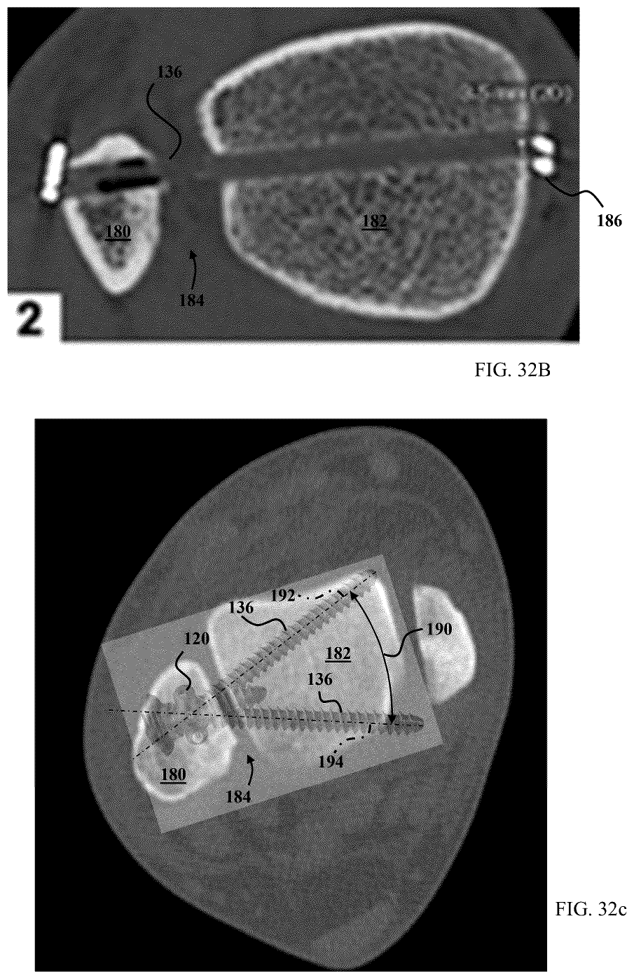

By way of example, FIG. 32A depicts a cross-section of a fibula 180 spaced apart from a tibia 182 by a syndesmotic joint 184. In a Type C2 fracture, the syndesmotic joint 184 is torn. Accordingly, in addition to the fibula 180, the syndesmotic joint 184 must also be stabilized when repairing a Type C2 fracture. FIG. 32b depicts the cross-section of the fibula 180 and tibia 182 with a screw 136 extending from the lateral side of the fibula 180 through the fibula 180, through the syndesmotic joint 184, and through the tibia 182 to engage a bone plate 186 positioned on the interior side of the tibia 182. The bone plate 186 is included in the kit 100. While this orientation of the screw 136 is effective for certain configurations of syndesmotic tears, it is not the most desired for other configurations.

The geometry of the intramedullary nails in the kit 100 overcomes the foregoing issue by providing a wide range of syndesmosis fixation. By way of example, FIG. 32C shows the distal portion 120 overlaying the cross-section of the fibula 180 and tibia 182 of FIG. 32A. As depicted in FIG. 32C, due to the additional width in the anterior-posterior plane provided by the distal portion 120 a variable angle syndesmosis fixation is possible with the screws 136. Accordingly, syndesmosis fixation is enhanced. In some embodiments the maximum syndesmosis fixation angle (α) 190 between the longitudinal axis 192 and 194 of the screws 136 is between 10 and 50 degrees, preferably between 20 and 45 degrees, and most preferably about 40 degrees.

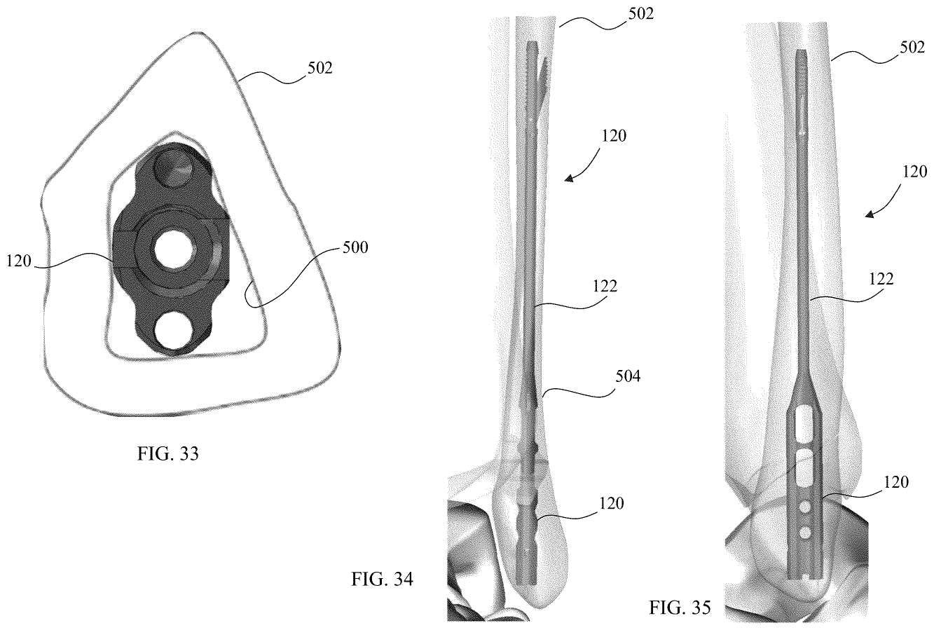

This configuration further provides a better anatomic match with a fibula compared to prior art intramedullary fibula nails. By way of example, FIG. 33 depicts the distal portion 120 of intramedullary fibula nail 102 positioned within an intramedullary canal 500 of a fibula 502. The distal portion 120 substantially fills the cross-section of the intramedullary canal 500. Additionally, as shown in FIG. 34, because the intramedullary fibula nail 102 is thinner than it is wide, interference with lateral cortex near the bend 504 in the fibula 502 is avoided. Moreover, the intramedullary fibula nail 102 is more flexible in the medial-lateral plane due to the relative thinness. At the same time, the increased width of the distal portion 120 as shown in FIG. 35 provides increased strength as well as rotational control.

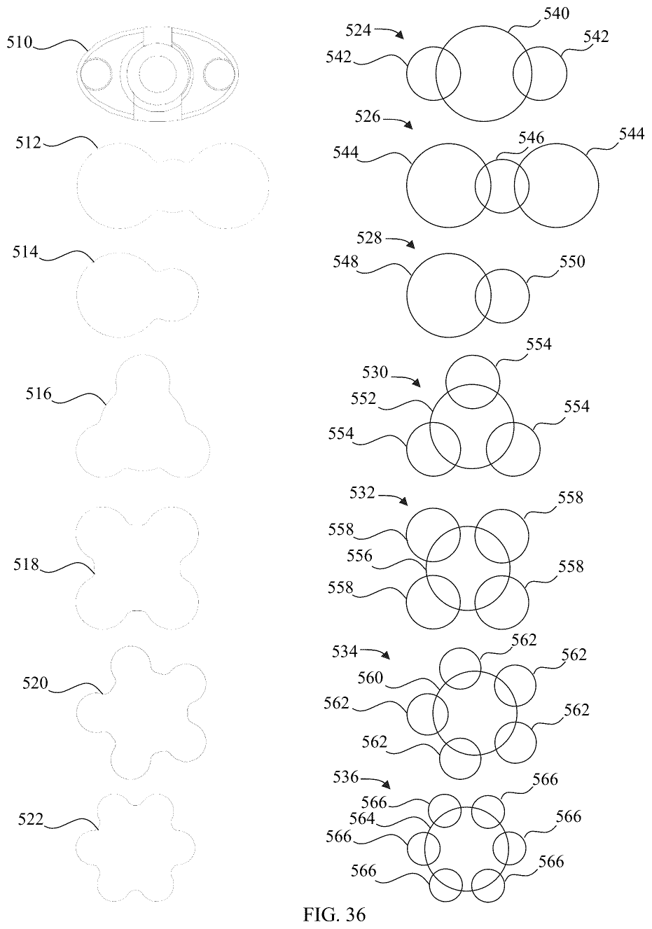

While the intramedullary fibula nails 102, 104, 106, 108, 110, and 112 are shown with distal portions exhibiting substantially identical cross-sectional shapes, in some embodiments one or more of the intramedullary fibula nails is provided additionally or alternatively with a different cross-sectional shape. Some alternative cross-sectional shapes are shown in FIG. 36. Each of the distal portion cross-sectional shapes 510, 512, 514, 516, 518, 520, and 522 are non-cylindrical and this provide at least some anti-rotation protection and, when combined with a cylindrical proximal portion, provide increased flexibility in the medial-lateral plane.

Moreover, a medullary canal in a fibula is easily prepared to receive any of the distal portion cross-sectional shapes 510, 512, 514, 516, 518, 520, and 522 using reamers and guide sleeves provided in the kit 100. In particular, each of the cross-sectional shapes can be at least substantially reamed using at least one large diameter reamer and at least one small diameter reamer as shown in schematics 524, 526, 528, 530, 532, 534, and 536 In particular, the schematics 524, 526, 528, 530, 532, 534, and 536 show relative diameters of reamers which can be used to form a cavity in a medullary canal of a fibula to accept the associated distal portion cross-sectional shape 510, 512, 514, 516, 518, 520, or 522, respectively.

Thus, as shown in schematic 524, a cavity for the distal portion cross-sectional shape 510 can be formed using a reamer with a large diameter 540 and at least one reamer with a small diameter 542. The schematic 524 is also used to provide the cavity for the distal portions in the intramedullary fibula nails 102, 104, 106, 108, 110, and 112 as shown in FIG. 1.

As shown in schematic 526, a cavity for the distal portion cross-sectional shape 512 can be formed using a reamer with at least one large diameter 544 and a reamer with a small diameter 546. As shown in schematic 528, a cavity for the distal portion cross-sectional shape 514 can be formed using a reamer with a large diameter 548 and a reamer with a small diameter 550. As shown in schematic 530, a cavity for the distal portion cross-sectional shape 516 can be formed using a reamer with a large diameter 552 and at least one reamer with a small diameter 554. As shown in schematic 532, a cavity for the distal portion cross-sectional shape 518 can be formed using a reamer with a large diameter 556 and at least one reamer with a small diameter 558. As shown in schematic 534, a cavity for the distal portion cross-sectional shape 520 can be formed using a reamer with a large diameter 560 and at least one reamer with a small diameter 562. As shown in schematic 536, a cavity for the distal portion cross-sectional shape 522 can be formed using a reamer with a large diameter 564 and at least one reamer with a small diameter 566.

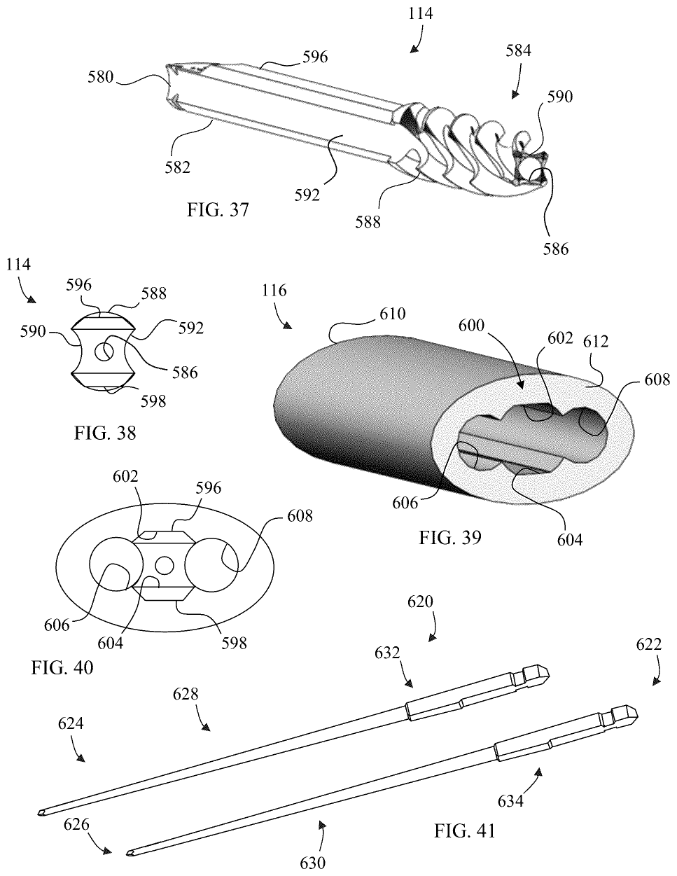

As is evident from schematics 524, 526, 528, 530, 532, 534, and 536, each large diameter portion of a prepared cavity overlaps at least one small diameter portion and each small diameter portion overlaps at least one large diameter portion. This is accomplished using at least one guide reamer such as the guide reamer 114 of FIG. 1. Referring to FIGS. 37 and 38, the guide reamer 114 includes a power coupling portion 580, a sleeve coupling portion 582, and a reaming guide portion 584. A guide bore 586 extends completely through the guide reamer 114.

The power coupling portion 580 is configured to couple with a rotating tool. In some embodiments the rotating tool is manually operated. In other embodiments the rotating tool is a power tool. The sleeve coupling portion 582 in some embodiments is configured as the power coupling portion 580. The reaming guide portion 584 is configured to ream a medullary canal of a fibula. The reaming guide portion 584 in this embodiment is threaded with a thread 588. Two side guide channels 590/592 extend along the entire length of the power coupling portion 580, the sleeve coupling portion 582, and the reaming guide portion 584. In some embodiments the kit 100 includes a guide reamer which is used to ream the “large” diameter while in some embodiments the guide reamer is used to ream the “large” diameter. In further embodiments, a plurality of guide reamers is provided with different diameters.

In the embodiment of FIGS. 37-38, the “large” diameter (see schematic 524) is provided by the guide reamer 114. Accordingly, the threads 588 have a diameter which corresponds to the large diameter 540 of FIG. 36. The side guide channels 590/592 are formed on circles having a diameter of the small diameter 542. The sleeve coupling portion 582 has an upper sleeve coupling surface 596 and a lower sleeve coupling surface 598. The upper sleeve coupling surface 596 and lower sleeve coupling surface 598 define a height and a shape of the sleeve coupling portion 582 which corresponds to a height and a shape of a coupling portion 600 of the guide sleeve 116 shown in FIG. 39.

The coupling portion 600 has an upper guide sleeve coupling surface 602 and a lower guide sleeve coupling surface 604 which define a height and a shape of the coupling portion 600 which corresponds to the height and the shape defined by the upper sleeve coupling surface 596 and lower sleeve coupling surface 598. The guide sleeve further defines two offset guide channels 606 and 608. The offset guide channels 606 and 608 are formed on circles having a diameter of the small diameter 542 and having origins which are spaced apart in a manner identical to the origins of the circles on which the side guide channels 590/592 are formed.

Consequently, the guide sleeve 116 is configured to non-rotatably couple with the guide reamer 114 by positioning the sleeve coupling portion 582 within the coupling portion 600 and moving the guide sleeve 116 toward the reaming guide portion 584 until the front surface 610 of the guide sleeve 116 contacts the rearmost portion of the thread 588. With the guide sleeve 116 non-rotatably coupled to the guide reamer 114, the guide sleeve 116 and the guide reamer 114 can be used to guide further reaming of the fibula. To this end, the kit 100 includes one or more side reamers such as the side reamers 620/622 shown in FIG. 41.

The side reamers 620/622 in this embodiment are identical. In other embodiments the side reamers have different lengths and/or different diameters. When different diameters are used, one set of the offset guide channels 606/608 and the associated side guide channels 590/592 is modified to match the further diameter. With reference to FIG. 41, the side reamers 620/622 include reaming portions 624/626, guide portions 628/630, and power coupling portions 632/634.

The power coupling portions 632/634 are configured to couple with a rotating device which in some embodiments is manually powered while in other embodiments a power tool is provided. The reaming portions 24/626 are configured to ream a medullary canal of a fibula and in some embodiments are threaded. The guide portions 628/630 are shaped complementary to the offset guide channels 606/608 and the side guide channels 590/592. In some embodiments, the maximum outer diameter of the reaming portions 624/626 and the guide portions 628/630 are identical.

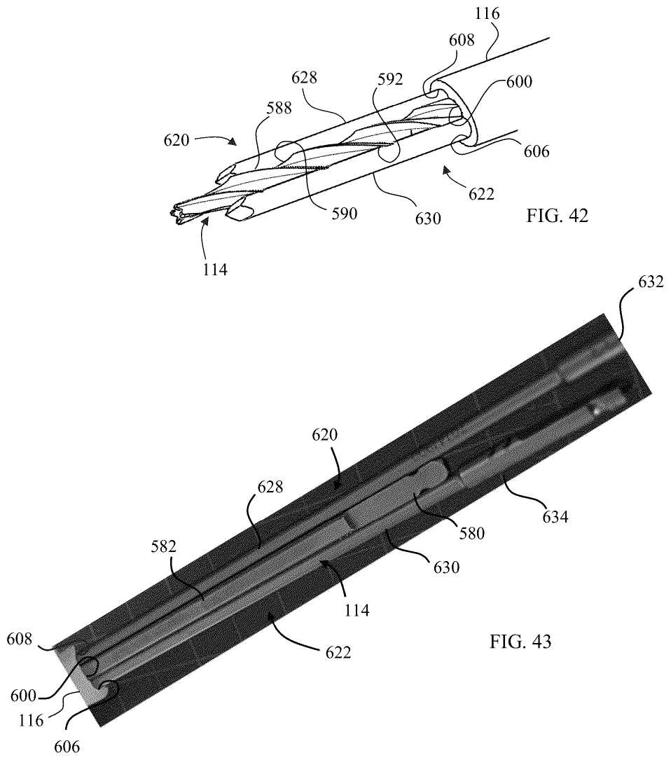

Accordingly, once the guide sleeve 116 is non-rotatably coupled to the guide reamer 114, the side reamers 620/622 are inserted into the guide sleeve 116. More specifically, one of the side reamers 620/622 is inserted into the offset guide channel 606 while the other of the side reamers 620/622 is inserted into the offset guide channel 608. While it is possible to use a single one of the side reamers 620/622 twice, a more stable construct is formed using two side reamers. As the side reamers 620/622 continue to be inserted into the guide channels 606/608, the guide portions 628/630 are guided by the respective one of the guide channels 606/608 and the side guide channels 590/592 as the reaming portions begin to ream the medullary canal adjacent to the reaming guide portion 584 of the guide reamer 114. Continued insertion of the side reamers 620/622 brings the guide portions 628/630 alongside the side guide channels 590/592 outside of the guide sleeve 116. The side guide channels 590/592 thus guide the side reamers 620/622 outside of the guide sleeve 116 until reaming has been completed resulting in the configuration shown in FIG. 42/43.

In FIG. 42, the thread 588 of the guide reamer 114 is extending outwardly of the guide sleeve 116 through the coupling portion 600. The guide portion 628 of the side reamer 620 is extending outwardly of the guide sleeve 116 through the offset guide channel 608. The guide portion 628 is further located partially within the side guide channel 590. The guide portion 630 of the side reamer 622 extends outwardly of the guide sleeve 116 through the offset guide channel 606. The guide portion 630 is further located partially within the side guide channel 592.

As shown in FIG. 43, the sleeve coupling portion 582 of the guide reamer 114 is extending outwardly of the guide sleeve 116 through the coupling portion 600. The guide portion 628 of the side reamer 620 extends outwardly of the guide sleeve 116 through the offset guide channel 608. The guide portion 630 of the side reamer 622 extends outwardly of the guide sleeve 116 through the offset guide channel 606.

While reaming using a guide reamer and two side reamers has been described, a single side reamer can be used either with the guide sleeve 114 or the guide sleeve 116. To accomplish this when using the guide sleeve 114, the side reamer is used with one offset guide channel to perform reaming, then removed and used with the other offset guide channel. When using the guide sleeve 116, after using the side reamer to ream the fibula the side reamer is removed. The guide sleeve 116 is then removed and rotated to position the offset guide channel for a second reaming operation with the side reamer. The same options are true with each of the distal portion cross-sectional shapes shown in FIG. 36 other than distal portion cross-sectional shape 514. Moreover, kits including one or more distal portions with one of the configurations of FIG. 36 include one or more guide sleeves modified to provide guidance for the associated cavity.

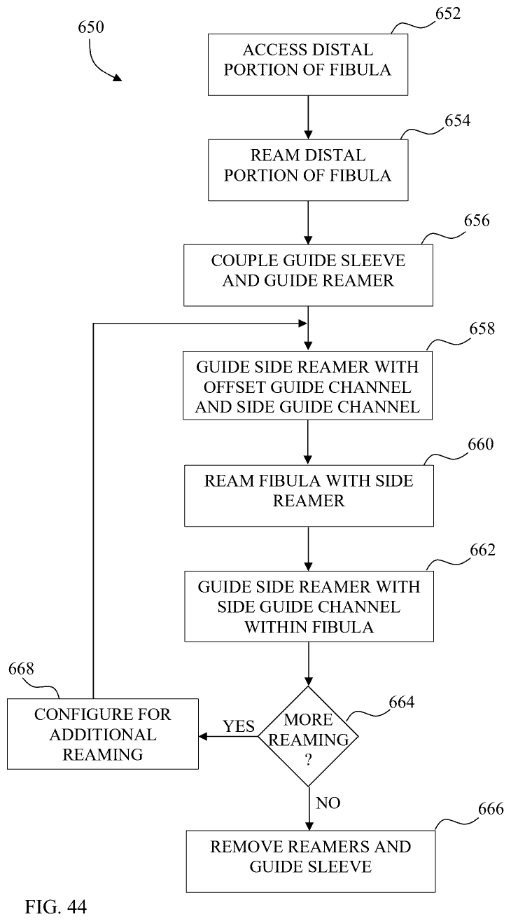

The kit 100 is used to prepare a fibula to receive an intramedullary fibula nail in accordance with the process 650 shown in FIG. 44. At block 652 a distal portion of the fibula is accessed. The medullary canal of the accessed fibula is then reamed using a guide reamer 114 at block 654. The guide reamer may be hand powered or driven by a power tool. In some embodiments, a guide wire such as a K-line is first implanted and is used to guide positioning of the guide reamer 114. In some embodiments, the guide wire is included in the kit 100. When the guide reamer 114 is inserted into medullary canal to the desired extent, a guide sleeve 116 or 118 or the like is coupled to the guide reamer by inserting the guide sleeve onto the guide reamer as described above such that the coupling portion 600 of the guide sleeve 616 couples with the sleeve coupling portion 582 to provide a non-rotational coupling. (Block 656). The insertion is terminated in some embodiments by abutment of the front surface 610 of the guide sleeve 116 (or 118 or the like) with the distal portion of the thread 588.

At block 658 a side reamer 620 is inserted into an offset guide channel (606 or 608) of the guide sleeve 116 (or 118 or the like) and guided by the offset guide channel and the associated side guide channel 590/592. Insertion continues until the side reamer 620 contacts the medullary canal at which time reaming with the side reamer commences at block 660. As the side reamer continues to ream the fibula, the guide portion 628 is guided within the guide sleeve 116 (118) by both the by the offset guide channel and the associated side guide channel, and within the fibula by the side guide channel until reaming reaches the desired depth (block 662). If all desired reaming has been accomplished at block 664, then at block 666 the reamers and guide sleeve are removed.

If at block 664 additional reaming is desired, then at block 668 the system is configured for additional reaming. In some embodiments, this simply entails selecting an additional side reamer and then proceeding to block 658. In other embodiments, block 668 includes removing the previously inserted side reamer and preparing to use the side reamer in another offset guide channel of the guide sleeve. In further embodiments, the guide sleeve and the side reamer are removed and the guide sleeve is re-oriented on the guide reamer before proceeding to block 658. The process continues until no more reaming is desired at block 664 and the method ends at block 666.

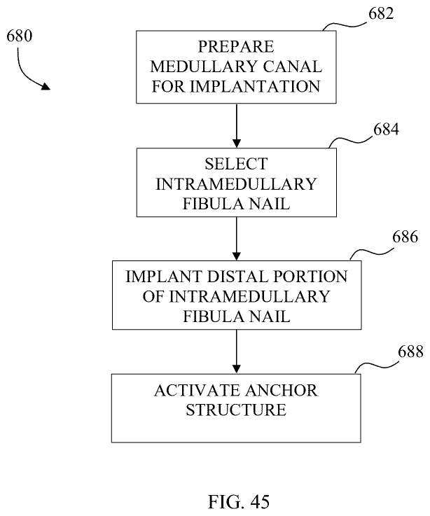

The kit 100 thus provides for a versatile approach to preparing a fibula to receive an intramedullary fibula nail. The kit 100 further allows for a versatile implantation of the intramedullary fibula nail using the process 680 of FIG. 45. In FIG. 45, the process 680 commences by preparing a medullary canal of a fibula to receive an implant at block 682.

The preparation of block 682 includes identification of the particular intramedullary fibula nail which is to be implanted. For example, while the intramedullary fibula nail 110 includes a proximal portion 432 which includes a reamer portion 442, the intramedullary fibula nail 102 does not. Moreover, while the longitudinal axis 151 of the proximal portion 122 of the intramedullary fibula nail 102 is aligned with the longitudinal axis 149 of the distal portion 120 in the intramedullary fibula nail 102, the longitudinal axis 286 of the distal portion 280 is offset from a longitudinal axis 288 of the proximal portion 282 in the intramedullary fibula nail 106. Accordingly, even when performing the process 650 of FIG. 44 in block 682, preparation of the medullary canal of a fibula differs depending upon the particular intramedullary fibula nail to be used.

Once the medullary canal of the fibula is prepared at block 682, the associated intramedullary fibula nail is selected at block 684. As discussed above, each of the intramedullary fibula nails 102, 104, 106, 108, 110, and 112 include a distal portion that is non-cylindrical and in some instances defines a first maximum width in a medial-lateral plane and a second maximum width in an anterior-posterior plane, wherein the second maximum width is larger than the first maximum width. Additionally, the further distal portion cross-sectional shapes described above are likewise non-cylindrical. Accordingly, regardless of the intramedullary fibula nail selected from the kit 100, the intramedullary fibula nail will have a non-cylindrical geometry.

The distal portion of the selected non-cylindrical intramedullary fibula nail is then implanted in the prepared medullary canal of the fibula at block 686. Depending upon the selected intramedullary fibula nail, implantation of the proximal portion of the intramedullary fibula nail may also effected at block 686. Implantation of the proximal portion 432 of the intramedullary fibula nail 110, however, occurs later in the process. In one embodiment, implantation of the distal portion of the selected non-cylindrical intramedullary fibula nail includes anchoring the distal portion using at least one screw.

In any event, following the implantation of at least the distal portion of the intramedullary fibula nail, at block 688 the anchor structure is activated. In some embodiments, this is accomplished using an activation suture. In the embodiment of intramedullary fibula nails 108 and 110, activation is effected by rotation of the anchor structure(s). In the intramedullary fibula nail 112, activation is accomplished by forcing the anchor structure proximally.

The described kit and process thus provide one or more intramedullary fibula nails which exhibit increased resistance to rotation along with an enhanced anatomic fit. A simplified fibular medullary canal preparation process is provided by the use of the disclosed guide sleeves and guide reamers. While the disclosure has been illustrated and described in detail in the drawings and foregoing description, the same should be considered as illustrative and not restrictive in character. It is understood that only the preferred embodiments have been presented and that all changes, modifications and further applications that come within the spirit of the disclosure are desired to be protected. By way of example, while the guide reamer 114 and the guide sleeve 116 in the kit 100 are provided as separate components, in some embodiments the kit 100 additionally or alternatively includes a combined guide reamer and sleeve.

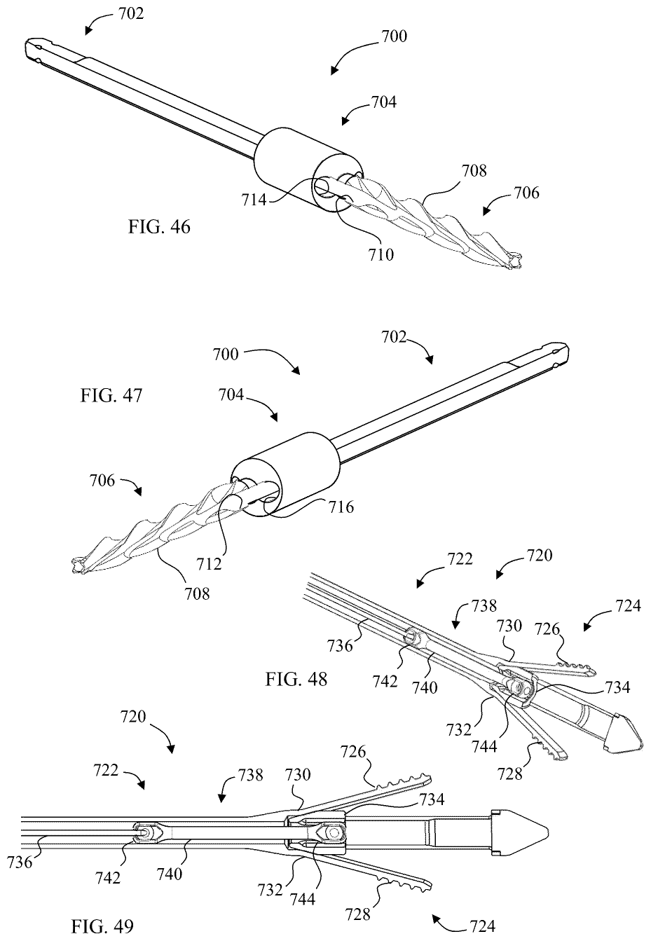

One such combined guide reamer and sleeve 700 is shown in FIGS. 46-47.

The combined guide reamer and sleeve 700 includes a power coupling portion 702, a sleeve guide portion 704, and a reaming guide portion 706. A thread 708 is provided along the reaming guide portion 706. Side guide channels 710/712 extend from the power coupling portion 702, through the sleeve guide portion 704 and the reaming guide portion 706. The integrally formed sleeve guide portion 704 defines offset guide channels 714/716. Thus, with the exception of being integrally formed, the combined guide reamer and sleeve 700 provide all of the functionality of the guide reamer 114 and the guide sleeve 116 with the exception that each side guide channel has an associated offset guide channel.

Additionally, one or more of the intramedullary fibula nails of FIG. 1 is modified or provided in a form which provides continuous tension to the suture activated anchoring structure. By way of example, FIG. 47-48 depict an embodiment of an intramedullary fibula nail 720 that includes a proximal section 722 with a suture activated anchoring structure 724. The suture activated anchoring structure 724 includes two serrated arm portions 726/728 and associated pivot regions 730/732. A wedge structure 734 is activated using a suture 736. The wedge structure 734 and suture 736, however, are joined by a nitinol tensioner 738 including a shank portion 740 and two coupling portions 742/744.

In operation, activation of the suture 736 causes the nitinol tensioner 738 to stretch resulting in continuous tension to the wedge structure 734 even after the suture is clamped with the suture activated anchoring structure 724 activated. Consequently, any suture stretch/creep is compensated by the nitinol tensioner 738 to ensure secure anchoring of the intramedullary fibula nail 720. The diameter of the nitinol tensioner 738 shank portion 740 is selected to achieve a desired stretch for an associated suture tension.

1. An intramedullary fibula nail kit, comprising:
an intramedullary fibula nail including
a proximal portion configured to be positioned within a medullary canal of a fibula, and
a distal portion configured to be positioned within the medullary canal of the fibula,
wherein
the distal portion defines a non-cylindrical geometry.

2. The intramedullary fibula nail kit of claim 1, wherein:
the proximal portion is cylindrically shaped;
the distal portion defines a first maximum width in a medial-lateral plane and a second maximum width in an anterior-posterior plane;
the second maximum width is larger than the first maximum width; and
the second maximum width is larger than a maximum diameter of the proximal portion.

3. The intramedullary fibula nail kit of claim 2, wherein:
the distal portion includes a neck portion which tapers to the proximal portion when the intramedullary fibula nail is implanted.

4. The intramedullary fibula nail kit of claim 3, wherein:
the proximal portion defines a proximal portion longitudinal axis;
the distal portion defines a distal portion longitudinal axis; and
the distal portion longitudinal axis is not aligned with the proximal portion longitudinal axis in the anterior-posterior plane when the intramedullary fibula nail is implanted.

5. The intramedullary fibula nail kit of claim 3, further comprising:
at least one threaded anchoring structure configured to anchor the intramedullary fibula nail,
wherein
the proximal portion defines a proximal portion longitudinal axis;
the at least one threaded anchoring structure defines an activated anchoring longitudinal axis: and
the activated anchoring longitudinal axis is parallel to the proximal portion longitudinal axis.

6. The intramedullary fibula nail kit of claim 5, wherein:
the activated anchoring longitudinal axis is coaxial with the proximal portion longitudinal axis; and
the proximal portion is configured to slidingly engage the distal portion.

7. The intramedullary fibula nail kit of claim 5, wherein:
the activated anchoring longitudinal axis is not coaxial with the proximal portion longitudinal axis; and
the at least one threaded anchoring structure is configured to extend outwardly from the distal portion.

8. The intramedullary fibula nail kit of claim 3, the intramedullary fibula nail further including:
at least one serrated anchoring structure configured to anchor the intramedullary fibula nail,
wherein
the proximal portion defines a proximal portion longitudinal axis;
the at least one serrated anchoring structure defines at least one activated anchoring longitudinal axis: and
the at least one activated anchoring longitudinal axis is parallel to the proximal portion longitudinal axis.

9. The intramedullary fibula nail kit of claim 8, further comprising:
at least one rod configured to extend outwardly from the distal portion and to support the at least one serrated anchoring structure.

10. The intramedullary fibula nail kit of claim 3, the intramedullary fibula nail further including:
at least one suture activated anchoring structure; and
an activation suture operably connected to the at least one suture activated anchoring structure.

11. The intramedullary fibula nail kit of claim 10, wherein:
the activation suture is operably connected to the at least one suture activated anchoring structure through a wedge component.

12. The intramedullary fibula nail kit of claim 10, wherein:
the activation suture is operably connected to the at least one suture activated anchoring structure through a wedge component.

13. The intramedullary fibula nail kit of claim 10, wherein:
the at least one suture activated anchoring structure is pivotably connected to the proximal portion through at least one pivot pin.

14. The intramedullary fibula nail kit of claim 13, wherein:
the at least one suture activated anchoring structure comprises two suture activated anchoring structures, each of the two suture activated anchoring structures pivotably connected to the proximal portion through a respective one of the at least one pivot pin.

15. The intramedullary fibula nail kit of claim 10, further comprising:
a retraction suture operably connected to the at least one suture activated anchoring structure,
wherein
the proximal portion defines a proximal portion longitudinal axis, and
the retraction suture is configured to bias at least a portion of the at least one suture activated anchoring structure in a direction toward the proximal portion longitudinal axis when the retraction suture is activated.

16. The intramedullary fibula nail kit of claim 14, further comprising:
a guide reamer including a sleeve coupling portion and at least one side guide channel;
a guide sleeve including a central coupling portion configured to non-rotatably couple with the sleeve coupling portion of the guide reamer, the guide sleeve including at least one offset guide channel; and
at least one offset reamer,
wherein
the proximal portion defines a proximal portion longitudinal axis, and
the at least one offset guide channel and the at least one side guide channel are configured to guide the at least one offset reamer within the guide sleeve and the at least one side guide channel is configured to guide the at least one offset reamer outside of the guide sleeve.

17. The intramedullary fibula nail kit of claim 16, wherein:
the distal portion defines a suture cross-over region;
the intramedullary fibula nail kit includes a clamping screw; and
the clamping screw is configured to clamp the activation suture within the suture cross-over region.

18. The intramedullary fibula nail kit of claim 17, further comprising:
a plurality of screws,
wherein
the distal portion includes a plurality of elongated screw holes, and
the plurality of screws and the plurality of elongated screw holes are configured to provide a syndesmosis fixation angle of between 10 and 50 degrees.

19. A method of preparing a fibula for an intramedullary fibula nail, comprising:
accessing a distal portion of the fibula;
reaming the fibula through the accessed distal portion with a guide reamer including a sleeve coupling portion and a side guide channel;
non-rotatably coupling a central coupling portion of a guide sleeve to the sleeve coupling portion of the guide reamer;
inserting a side reamer into an offset guide channel of the non-rotatably coupled guide sleeve;
guiding the inserted side reamer with the side guide channel and the offset guide channel within the non-rotatably coupled guide sleeve; and
reaming the accessed distal portion with the inserted side reamer while guiding the inserted side reamer outside of the guide sleeve with the side guide channel.

20. A method of implanting an intramedullary fibula nail, comprising:
preparing a fibular medullary canal for implantation;
selecting an intramedullary fibula nail with a proximal portion, and a distal portion that exhibits a non-cylindrical geometry; and
implanting the distal portion within the fibular medullary canal.

21. The method of claim 20, wherein:
the distal portion defines a first maximum width in a medial-lateral plane and a second maximum width in an anterior-posterior plane;
the second maximum width is larger than the first maximum width; and
the method further comprises activating an anchoring structure after implanting the distal portion.
